(19) United States
(12) Patent Application Publication
(10) Pub. No.: US 2007/0129636 A1
(43) Pub. Date: Jun. 7, 2007

(54) Vital sign monitor utilizing historic patient data

(76) Inventors: Bruce A. Friedman, Tampa, FL (US); John W. Booth, Tampa, FL (US); Richard Medero, Tampa, FL (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/292,037

(22) Filed: Dec. 1, 2005

(51) Int. Cl. A61B 5/02; A61B 5/00

(52) U.S. Cl. 600/481; 600/301; 600/485; 600/549; 600/323; 600/300

(57) ABSTRACT

A vital sign monitoring system that can be used with multiple patients and utilizes historic patient data information for the patient to optimize the process of obtaining current vital sign measurements. Each patient is identified with a unique patient identification device that is automatically detected by the vital sign monitor. The vital sign monitor communicates with a medical records database and obtains historic patient data information for the patient identified by the patient identification device. The historic patient data information can be utilized by the vital sign monitor to set alarm limits for the vital sign measurements and automatically control the operation of an NIBP monitor for the specific patient.

### BACKGROUND OF THE INVENTION

The present invention generally relates to a vital sign monitor that can be used for collecting vital sign measurements, including non-invasive blood pressure readings, from one or more patients. More specifically, the present invention relates to a vital sign monitor that can obtain historic patient data measurements for each patient and optimize the operation of the vital sign monitor based upon the historic measurements.

Blood pressure is a vital sign that is typically measured on patients in medical settings. Blood pressure readings are most often taken using non-invasive blood pressure cuffs attached to the upper arm of the patient. The cuff is operatively connected to a blood pressure monitor, which receives readings from the cuff, analyzes the readings using various predetermined algorithms, and displays measurement values associated with the blood pressure of the patient. The blood pressure measurements may be taken as part of more a comprehensive vital signal monitoring process that can also collect other important data from a patient, such as temperature and heart rate.

In many applications, the vital sign monitor is transported between multiple patients and the vital sign measurements taken from each patient are recorded by medical personnel. The measured vital sign data can be either automatically or manually entered into a hospital information system (HIS) such that the measurement data can be accessible by other personnel in the hospital and forms part of the patient's record.

Each time the vital sign monitor is utilized with a new patient, the blood pressure cuff is placed around the patient's arm and is inflated to an initial inflation pressure. Since the vital sign monitor is used with multiple patients, the monitor does not have access to any historic information regarding the patient. Thus, the vital sign monitor inflates the blood pressure cuff to a standard initial inflation pressure. In many cases, the initial inflation pressure will be well above the systolic pressure for the patient, thereby requiring the blood pressure cuff to be deflated in a series of steps prior to the cuff pressure reaching the systolic pressure. The over-inflation of the blood pressure cuff results in both patient discomfort and an increased amount of time required to take a blood pressure reading for the patient.

When the vital sign monitor is utilized with multiple patients, it is also difficult to provide alarm limits for the vital signs being obtained from each patient, since historic data for each of the patients is not readily available without the medical personnel consulting historic data charts at the patient's location. Thus, the use of a vital sign monitor that travels between multiple patients eliminates the ability to provide preset alarm limits for different vital signs based on the patient's condition, such as patient temperature or heart rate.

It is therefore desirable to provide a vital sign monitor that can utilize historic data for the patient to modify the operation of the vital sign monitor and set alarm limits for various vital sign parameters.

### SUMMARY OF THE INVENTION

The following describes a method and apparatus for obtaining vital sign information from one or more patients utilizing a single vital sign monitoring device. Preferably, the vital sign monitoring device includes at least a non-invasive blood pressure (NIBP) monitoring system, as well as components for obtaining the temperature and heart rate from the patient. The blood pressure cuff of the NIBP monitoring system is selectively inflated and deflated. During the deflation of the blood pressure cuff from an initial, target inflation pressure, oscillometric pulses are detected and the processor within the vital sign monitor calculates the blood pressure based upon the oscillometric pulses.

The method and system for optimizing the operation of the vital sign monitor includes providing each patient with a unique patient identification device that can be read by the vital sign monitor. As an illustrated example, the patient identification device can include a bar code attached to the patient that can be automatically read by a bar code reader of the vital sign monitor. Alternatively, the patient's identification device can be an RF tag that can be detected by a RF detector included within the vital sign monitor.

Once the vital sign monitor has obtained the patient identification information from the patient, such as through the use of the bar code scanner, the vital sign monitor communicates with an electronic medical records database that includes historic information for the patient identified by the patient identification information. Preferably, the medical records database includes historic patient data measurements and other medically relevant information for the patient. This includes but is not limited to patient vital sign data and patient demographic data (e.g. weight, height, age, diagnosis, gender). The historic measurements and medical information is uploaded into the vital sign monitor such that the vital sign monitor can optimize the operation of the vital sign measurement processes based upon the historic data.

Upon receiving the historic patient information, the vital sign monitor sets alarm limits for various vital sign parameters being obtained from the patient. As an example, maximum and minimum thresholds can be set for the patient's heart rate, oxygen saturation and temperature based upon reference values determined from past measurement cycles.

In addition to setting alarm limits, the vital sign monitor can automatically adjust the operation of the vitals sign monitor based upon measurements taken during preceding measurement cycles. Specifically, for blood pressure measurements the vital sign monitor can adjust the initial inflation pressure based upon the systolic pressure measured over several prior measurement cycles. The adjustment of the initial inflation pressure eliminates over-inflation of the pressure cuff and optimizes the blood pressure measurement cycle.

Once the initial inflation pressure has been adjusted based on patient-specific past measurements, the vital sign monitor inflates the blood pressure cuff to the initial inflation pressure. After the initial inflation pressure has been reached, the blood pressure cuff is deflated in a series of steps. During each step, oscillometric pulses are measured such that the NIBP monitoring system can determine the blood pressure for the patient.

The historic patient data could be used in a similar manner to change other features of the vital signs monitor, including but not limited to changing filter settings on the blood pressure or pulse oximeter monitors or adjusting analysis of vital sign measurements based on patient demographic data.

Once the blood pressure and other vital signs have been obtained from the patient, the vital sign monitor communicates the current blood pressure readings and other vital sign measurements back to the medical records database such that the patient's records can be updated. After the vital signs from a patient have been measured, the vital sign monitor can be taken to another patient and the patient automatically identified. Once the patient is automatically identified, the vital sign monitor obtains historic information for the patient and optimizes the vital sign measurement cycle as described.

### BRIEF DESCRIPTION OF THE DRAWINGS

The drawings illustrate the best mode presently contemplated for carrying out the invention. In the drawings:

FIG. 1 is an illustrative view of the vital sign monitor having communication capability with a medical records database and a bar code reader used to identify a patient;

FIG. 2 illustrates oscillometric data, including step deflate and oscillation pulse amplitudes, derived using the NIBP monitor of the vital sign monitoring system shown in FIG. 1; and

FIG. 3 is a flow chart illustrating one example of the steps utilized to obtain patient data from the medical records database and modifying the operation of the vital sign monitor.

### DETAILED DESCRIPTION OF THE INVENTION

In the embodiment of the invention described in detail below, an apparatus, system and method for collecting vital sign information from a plurality of patients is provided. It should be understood that the drawings and specification are to be considered an exemplification of the principles of the invention, which is more particularly defined in the appended claims. For example, although specific embodiments of the blood pressure cuff and vital sign monitor including non-invasive blood pressure monitoring capabilities are depicted in the figures, it will be recognized that different vital sign monitoring equipment can be utilized while employing the principles of the present invention. In addition, although the figures depict particular steps related to the method of the invention, it will be recognized that alternative, equivalent steps or procedures may be employed within the principles of the claimed invention.

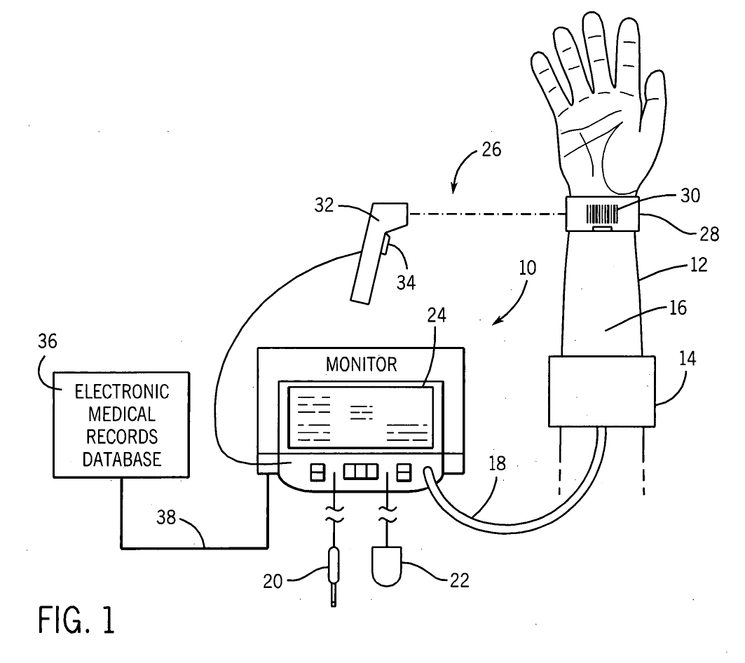

Referring first to FIG. 1, a vital sign monitor 10 is shown. The vital sign monitor 10 is preferably a mobile device that can be moved between multiple patients by medical personnel to obtain vital sign measurements from each of the patients. In the embodiment of the invention illustrated, the vital sign monitor 10 includes the ability to obtain vital sign measurements from a plurality of individual patients, such as blood pressure readings, temperature readings, heart rate and blood oxygen saturation from the patient 12.

As illustrated, the vital sign monitor 10 includes a blood pressure cuff 14 that can be placed on an arm 16 of the patient 12 to obtain blood pressure measurements in a manner to be described below. The blood pressure cuff 14 is connected to the vital sign monitor 10 by an air hose 18. The vital sign monitor 10 is coupled to a supply of pressurized air such that the vital sign monitor 10 can selectively inflate and deflate the pressure cuff 14 through the air hose 18.

In addition to the pressure cuff 14, the vital sign monitor 10 includes an electronic thermometer 20 for obtaining the patient temperature and a pulse oximeter probe 22 that is operable to obtain both heart rate and blood oxygen saturations from the patient. Preferably, vital sign monitor 10 includes a display 24 that visually presents the obtained vital sign information. The vital sign monitor 10 is automatically operable to obtain the blood pressure measurement, temperature, heart rate and blood oxygen saturation from the patient 12 once the blood pressure cuff 14, thermometer 20 and probe 22 have been placed on the patient. The operation of the vital sign monitor 10 to obtain this information is well known

In the embodiment of the invention illustrated in FIG. 1, the vital sign monitor 10 includes detection means 26 for providing patient identification information to the vital sign monitor 10. Specifically, the detection means includes the unique patient identification device 28 placed on each individual patient. In the specific embodiment illustrated in FIG. 1, the patient identification device 28 is a bracelet including a bar code 30. Although a pre-printed bar code 30 is shown in the illustrated embodiment, the patient identification device 28 could be a radio frequency tag or other means for uniquely identifying the patient 12.

In the illustrated embodiment, the detection means 26 also includes a bar code reader 32 coupled to the vital sign monitor 10. The bar code reader 32 can be actuated using trigger 34 to electronically read the bar code 30 attached to the patient 12. Since each patient includes a unique bar code 30, the bar code reader 32 can automatically and electronically identify the patient 12 and relay the identification information to the vital sign monitor 10 using either a hard-wired communication link or a wireless communication link between the vital sign monitor 10 and the bar code reader 32.

In the embodiment of the invention illustrated in FIG. 1, the vital sign monitor 10 is in communication with an electronic medical records database 36. As an example, the electronic medical records database can be part of a hospital information system (HIS) and configured to store historic patient data information for each patient within the hospital. The medical records database 36 can include not only vital sign information, but other medically relevant information relating to each of the patients. As an example, the medical records database 36 can include information relating to the patient's age, past diagnosis information, such as if the patient is diabetic, and past treatment information.

In the embodiment of the invention illustrated in FIG. 1, the vital sign monitor 10 is in communication with the medical records database 36 over a communication link 38. The communication link 38 can be either a hard-wired communication link or a wireless communication link between the vital sign monitor 10 and the medical records database 36.

As described previously, the vital sign monitor 10 includes a non-invasive blood pressure (NIBP) monitor that controls the inflation and deflation of the pressure cuff 14 to obtain blood pressure information from the patient. Specifically, the blood pressure cuff 14 includes a transducer that is used to sense pressure oscillations in the cuff that are generated by pressure changes in the brachial artery under the blood pressure cuff 14. The electrical signals from the pressure transducer are obtained by the vital sign monitor 10 and are used by well-known algorithms operating in the vital sign monitor 10 to calculate the patient's blood pressure.

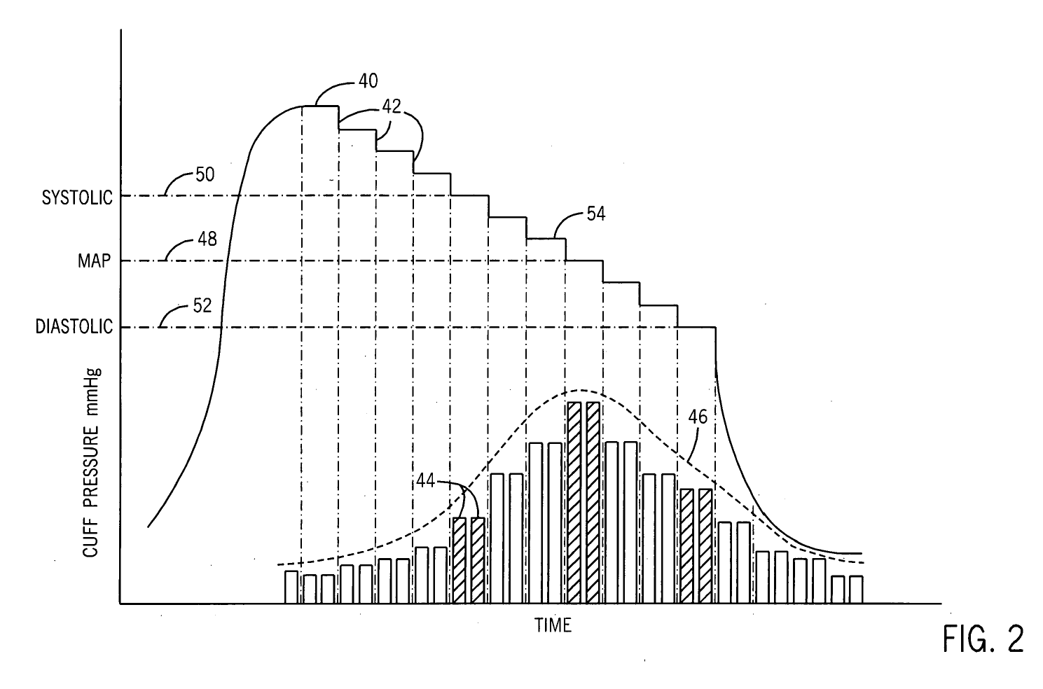

During normal operation of the vital sign monitor 10, the blood pressure cuff 14 is placed on the patient 12, typically around the patient's arm 16 over the brachial artery. At the inception of the measuring cycle, the blood pressure cuff 14 is inflated to an initial inflation pressure that fully occludes the brachial artery, i.e., prevents blood from flowing through the brachial at any point in the heart's cycle. In FIG. 2, the initial inflation pressure is illustrated by reference numeral 40.

After the blood pressure cuff has been inflated to the initial inflation pressure 40, the pressure cuff is deflated in a series of pressure steps 42. After each pressure step 42, the NIBP monitoring system detects and records one or more pressure oscillations 44 for the current cuff pressure. The pressure transducer measures the internal cuff pressure and provides an analog signal characterizing the blood pressure oscillations. The peak values of the complex signals are determined within the vital sign monitor.

As the measurement cycles progress, the peak amplitude of the blood pressure complexes generally become monotonically larger to a maximum and then become monotonically smaller as the cuff pressure continues toward full deflation, as illustrated by the general bell curve 46 in FIG. 2. The oscillometric measurements are used by an algorithm operating within the vital sign monitor to calculate the mean arterial pressure (MAP) 48, the systolic pressure 50 and the diastolic pressure 52 in a known manner.

As can be understood in FIG. 2, the initial inflation pressure 40 for the blood pressure cuff must exceed the systolic pressure 50 for the system and method of NIBP monitoring to function effectively. In past embodiments of a vital sign monitor that can be used with multiple patients, the initial inflation pressure 40 is a standard value. Since the vital sign monitor is utilized with multiple patients, the initial inflation pressure 40 is selected such that the initial inflation pressure 40 will be above the typical systolic pressure 50 for most patients. Typically, the initial inflation pressure 40 is selected at a relatively high value such that it will exceed the systolic pressure 50 for most patients.

FIG. 2 illustrates an embodiment in which the initial inflation pressure 40 is significantly higher than the systolic pressure 50 for the particular patient. In this operational example, the pressure within the blood pressure cuff must be decreased a significant number of pressure steps 42 before the cuff pressure 54 reaches the systolic pressure 50. The over inflation of the blood pressure cuff results in the patient experiencing discomfort due to unnecessarily high cuff pressures and prolonged occlusion of the brachial artery. Further, the over inflation of the blood pressure cuff increases the overall time required to take a blood pressure reading from the patient due to the numerous pressure steps 42 required before the cuff pressure reaches the systolic pressure 48.

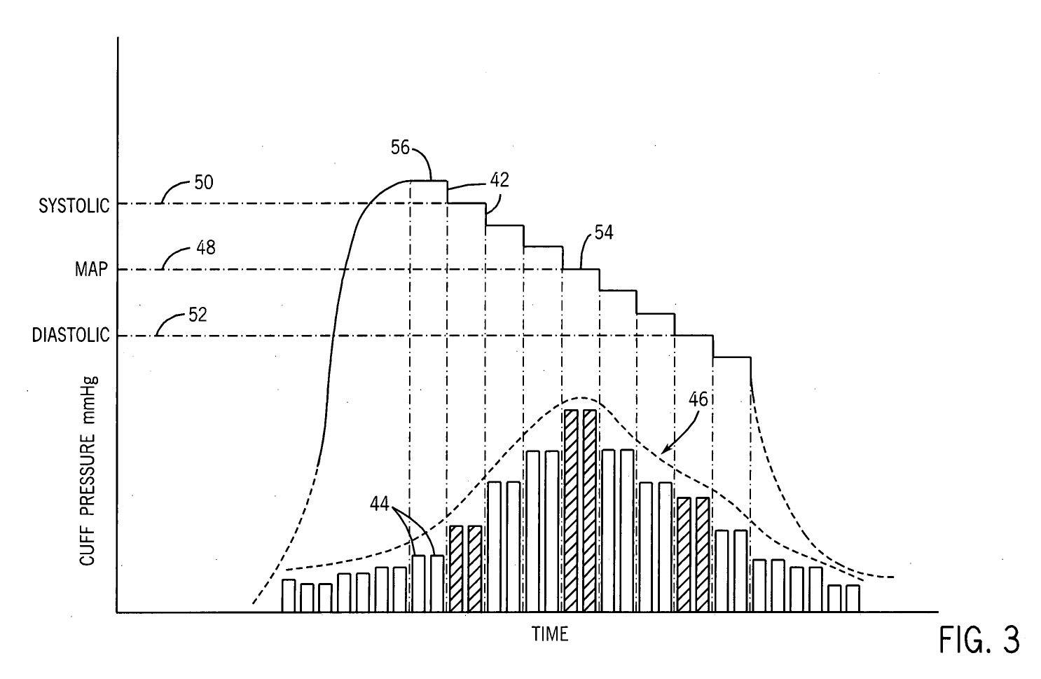

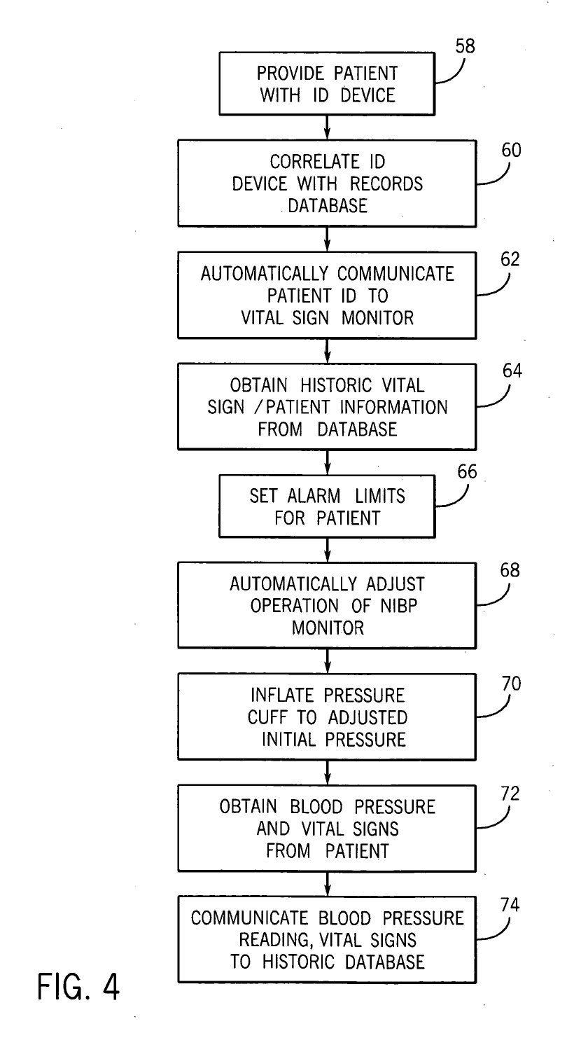

Referring now to FIG. 3, thereshown is a preferred method of operating the NIBP system in accordance with the present invention. As illustrated, the initial inflation pressure 56 is selected much closer to the systolic pressure 50 for the patient such that only a single pressure step 42 is required to deflate the cuff pressure 54 to the systolic pressure 50. In this embodiment, the cuff is no longer over-inflated and the time required to obtain the blood pressure measurements has been decreased.

In order to achieve the optimized inflation pressure 56 shown in FIG. 3, the vital sign monitor 10 calculates the initial inflation pressure for the specific patient based upon historic blood pressure measurements taken for the patient 12 that are stored in the medical records database 36 shown in FIG. 1. Specifically, the vital sign monitor 10 accesses the stored historic patient data measurements from the medical records database 36 for the specific patient and utilizes an algorithm to select the initial inflation pressure such that the initial inflation pressure is based upon the historical vital sign information obtained from the patient, rather than a standard inflation pressure utilized for all patients.

In addition to optimizing the initial inflation pressure, the vital sign monitor 10 of the present invention utilizes additional historic patient data information obtained from the medical records database 36 to set alarm parameters and tailor the operation of the vital sign monitor 10 for the specific patient 12.

The operation of the vital sign monitor 10 will be described with reference to FIG. 4. Initially, each individual patient is provided with a unique, patient identification device in step 58. As described previously, the patient shown in FIG. 1 is provided with an identification bracelet having a unique bar code 30. However, other patient identification devices, such as an RF tags, can be utilized while operating within the scope of the present invention.

Once the patient has been given an identification device, the identification device is correlated with patient records stored in the medical records database 36, as indicated in step 60. The correlation of the patient identification device with the patient's record in the medical records database allows patient information to be retrieved from the database and utilized by the vital sign monitor 10.

After each patient has been assigned a patient identification device, the patient identification information from the patient identification device is automatically communicated to the vital sign monitor in step 62. In the embodiment of the invention illustrated in FIG. 1, the patient identification information is included on a bar code 30 and is read by the bar code reader 32. However, other automatic methods of obtaining patient identification information are contemplated as being within the scope of the present invention.

Referring back to FIG. 4, after the patient identification information has been obtained by the vital sign monitor 10, the vital sign monitor communicates to the medical records database 36 to obtain historic patient data measurements and patient information from the medical records database 36, as illustrated in step 64. The historic data stored in the electronic medical records database 36 can include information relating to the past measurements obtained by the vital sign monitor 10. This information can include past blood pressure readings, past heart rate measurements, past patient temperatures and other information that may be related to the steps required to obtain the vital sign information from the patient. In addition to the past vital sign measurements, other relevant medical information, such as the patient's age, medical condition, weight or other similar information can be obtained by the vital sign monitor in step 64. As previously indicated, the vital sign monitor 10 can obtain the information from the medical records database using either a wired or wireless communication link.

Since the vital sign monitor 10 is contemplated as being used with multiple different patients, the vital sign monitor 10 can obtain patient-specific information from the medical records database 36 and utilize the patient-specific information to optimize the steps required to obtain the vital signs from the patient. When the vital sign monitor 10 is moved to the next patient, patient-specific vital sign information can be obtained from the medical records database and utilized to optimize the vital sign measurement for the next patient.

Referring back to FIG. 4, once the historic information is received for the specific patient being monitored by the vital sign monitor, internal algorithms within the vital sign monitor can be used to set alarm limits for the specific patient, as illustrated in step 66. As an example, based upon the most recent heart rate measurements obtained for the patient, the algorithm within the vital sign monitor can set alarm limits for the heart rate based upon threshold levels both above and below the most recent measurements. Since heart rate is dependent upon the physical state, age and weight of the patient, the algorithm can use these other parameters, along with past measurements, obtained from the medical records database to set alarm limits for the patient. Further, since the medical diagnosis for the patient also affects acceptable heart rate levels, this information can also be used to set the alarm limits in step 66.

In step 68, the vital sign monitor 10 automatically adjusts the operation of the NIBP monitoring components to control the inflation/deflation of the pressure cuff 14. As discussed previously, it is highly desirable to select the initial inflation pressure for the blood pressure cuff based upon past blood pressure measurements for the patient. Specifically, it is desirable to select the initial inflation pressure as close to the systolic pressure as possible to avoid over inflation and to decrease the amount of time required to obtain a blood pressure measurement.

When selecting the initial inflation pressure, the vital sign monitor utilizes an algorithm that estimates the systolic pressure of the patient based upon at least one past measurement. Since the initial inflation pressure must be above the systolic pressure for proper operation of the NIBP monitoring system, the initial inflation pressure is selected a determined amount above the predicted systolic pressure for the patient. As can be understood, selecting the initial inflation pressure for the specific patient is a vast improvement over prior art systems that select the same initial inflation pressure for each patient.

As illustrated in step 70, the vital sign monitor inflates the pressure cuff to the adjusted initial inflation pressure. After the blood pressure cuff has been inflated to the initial inflation pressure, the cuff is deflated in the series of pressure steps to obtain the oscillometric information required to calculate the blood pressure for the patient. At the same time, the vital sign monitor 10 can also obtain other vital sign information from the patient, as illustrated in step 72. It is contemplated that the other vital sign information obtained by the vital sign monitor may include heart rate, temperature, blood-oxygen saturation and any other parameters that may be useful in monitoring a patient.

In addition to utilizing the historic patient data measurements to adjust the initial inflation pressure, the vital sign monitor can also utilize the historic patient data measurements and medical information for the patient to modify the algorithm utilized to calculate the blood pressure for the patient based upon the oscillations received from the blood pressure cuff. As an example, the NIBP algorithm used to calculate blood pressure may be adjusted based upon the patient's age or the disease state of the patient. As an example, the oscillations received from a patient having diabetes or another disease may be reduced as compared to oscillations received from a healthy patient having the same blood pressure. Thus, the vital sign monitor can not only modify the initial settings for the blood pressure measurement, but may also modify the actual algorithm used to determine the blood pressure measurements.

As the blood pressure and vital sign information is obtained from the patient, the vital sign monitor compares the vital sign information to the alarm limit set in step 66. If the vital sign information exceeds the alarm limits, an alarm can be generated to immediately indicate to medical personnel that the patient has significantly deviated from past medical readings. Since the vital sign monitor 10 is utilized with multiple patients, the ability to automatically set alarm limits for the specific patient based upon historic information from that specific patient provides additional benefits to medical personnel who are often required to monitor numerous patients during a given day.

As illustrated in FIG. 4, once the blood pressure reading and vital signs have been collected by the vital sign monitor, this current information is communicated to the electronic medical records database existing at the respective hospital or healthcare setting. Such information can be provided in real time and is useful in updating patient records and providing up-to-the-minute information to caregivers throughout the hospital. This is illustratively shown in step 74. The updated information is then utilized by the vital sign monitor the next time the vital signs for a new patient are measured.

It is important to note that different vital sign monitors can be utilized with the plurality of patients, since the vital sign monitor contacts the electronic medical records database to obtain historic information. Thus, since the patient information is not stored locally on the vital sign monitor and instead is stored at an accessible, remote location, different vital sign monitors can be utilized for monitoring the plurality of patients.

In the embodiment of the invention illustrated in FIG. 1, the medical records database 36 is shown located remotely from the vital sign monitor 10. As an example, the medical records database 36 may be the HIS system for the facility in which the patients are located. In the embodiment illustrated, the vital sign monitor 10 communicates to the medical records database 36 utilizing either a hard wire or wireless communication technique.

It is contemplated that the vital sign monitor 10 could include a limited medical records database within the physical housing of the vital sign monitor. If the medical records database is included within the actual vital sign monitor, the vital sign monitor would no longer need to contact the remotely located medical records database.

In a vital sign monitor including an internal medical records database, a patient record would be established for each patient during the initial vital sign measurement cycle. Each time the vital sign monitor is used with the same patient, the patient identification information received from the patient would be utilized to retrieve historic information stored within the internal medical records database. Since the internal database contained within the vital sign monitor would have limited storage capabilities, once a patient has been discharged or is no longer being monitored, that patient's information would be removed from the internal medical records database. The vital sign monitor 10 including an internal medical records database is contemplated as being particularly useful in small facilities that do not include any central medical records database. The internal medical records database would allow the vital sign monitor 10 to utilize past measurements from the patient to optimize the procedures required to obtain current vital sign measurements from the patient.

Various alternatives and embodiment are contemplated as being within the scope of the following claims particularly pointing out and distinctly claiming the subject matter regarded as the invention.

I claim:

1. A system for collecting vital sign measurements from one or more patients, the system comprising:
a vital sign monitor operable to obtain current vital sign measurements from each of the patients;
a means for obtaining patient identification information from the patient and providing the patient identification information to the vital sign monitor; and
a communication means for retrieving historic patient data information for the patient identified by the patient identification information,
wherein the operation of the vital sign monitor is adjusted based upon the historic patient data information retrieved for the patient.

2. The system of claim 1 wherein the patient identification information is detected automatically.

3. The system of claim 2 wherein the communication means comprises a unique bar code associated with each of the patients and a bar code reader associated with the vital sign monitor.

4. The system of claim 2 wherein the communication means comprises a unique radio frequency identification tag associated with each of the patients and a radio frequency receiver associated with the vital sign monitor.

5. The system of claim 1 wherein the vital sign monitor includes at least a blood pressure sensor for obtaining the blood pressure and heart rate of the patient.

6. The system of claim 5 wherein the blood pressure sensor comprises an inflatable and deflatable non-invasive blood pressure cuff arranged to be worn around a limb of the patient and operatively connected to the vital sign monitor to provide blood pressure measurement data to the vital sign monitor.

7. The system of claim 5 wherein the vital sign monitor includes at least a pulse oximeter sensor for obtaining the oxygen saturation and heart rate of the patient.

8. The system of claim 5 wherein the vital sign monitor includes at least a temperature sensor for obtaining the temperature of each patient and a heart rate monitor for obtaining the heart rate of each patient.

9. The system of claim 1 further comprising a medical records database for storing the historic patient data measurements obtained from each of the patients, wherein the communication means is operable to download the current vital sign measurements obtained from the patient to the medical records database and retrieve the historic patient data measurements for each of the patients from the medical records database.

10. The system of claim 9 wherein the historic patient data measurements are retrieved from the medical records database based upon the patient identification information obtained from the patient.

11. The system of claim 9 wherein the vital sign monitor controls the method of analyzing the data from at least one of a blood pressure sensor, a pulse oximeter sensor or a temperature sensor based upon the historic patient data measurements and demographic data retrieved from the medical records database for the patient

12. The system of claim 10 wherein the communication means communicates with the historic database using wireless communication.

13. The system of claim 10 wherein the vital sign monitor sets alarm parameters for each patient based upon the historic patient data measurements retrieved from the medical records database.

14. The system of claim 6 wherein the vital sign monitor selects an initial inflation pressure for the blood pressure cuff based upon the retrieved historic patient data measurements for the patient.

15. The system of claim 9 wherein the medical records database is located remotely from the vital sign monitor.

16. The system of claim 9 wherein the medical records database is located within the vital sign monitor.

17. A method of obtaining current vital sign information from one or more patients utilizing a single vital sign monitor, the method comprising the steps of:
providing each patient with a unique, machine readable identification device;
obtaining patient identification information for the patient;
electronically communicating with a medical records database that includes stored, historic patient data information for each of the plurality of patients;
retrieving the historic patient data information for the patient based on the obtained patient identification information; and
controlling the operation of the vital sign monitor based on the historic patient data information for the patient to obtain current vital sign measurements for the patient.

18. The method of claim 17 wherein the vital sign monitor includes at least a blood pressure sensor for obtaining the blood pressure and heart rate of the patient

19. The method of claim 18 wherein the blood pressure sensor comprises an inflatable and deflatable non-invasive blood pressure cuff arranged to be worn around a limb of the patient and operatively connected to the vital sign monitor to provide blood pressure measurement data to the vital sign monitor.

20. The method of claim 19 wherein an initial inflation pressure for the blood pressure cuff is determined based upon the retrieved historic patient data information for the patient.

21. The method of claim 17 wherein the identification device for each patient is a unique bar code and the step of obtaining the patient identification information for each patient includes utilizing a bar code reader to read the bar code for the patient.

22. The method of claim 17 wherein the identification device for each patient is a unique radio frequency identification tag and the step of obtaining the patient identification information for each patient includes utilizing a radio frequency receiver associated with the vital sign monitor to detect the radio frequency identification tag for the patient.

23. The method of claim 17 wherein the medical records database is located remotely from the vital sign monitor and the step of retrieving the historic patient data information includes accessing the medical records database using wireless communication.

24. The method of claim 17 further comprising the steps of:
setting alarm parameters for at least one vital sign measurement to be obtained from the patient based upon the retrieved historic patient data measurement for the patient;
operating the vital sign monitor to obtain the current vital sign measurements from the patient; and
activating an alarm when the obtained vital sign measurement exceeds the alarm parameters.

25. The method of claim 24 wherein the vital sign monitor includes at least a blood pressure sensor for obtaining the blood pressure and heart rate of the patient.

26. The method of claim 25 wherein the blood pressure sensor comprises an inflatable and deflatable non-invasive blood pressure cuff arranged to be worn around a limb of the patient and operatively connected to the vital sign monitor to provide blood pressure measurement data to the vital sign monitor.

27. The method of claim 17 further comprising the step of
transferring the current vital sign measurements from the vital sign monitor to the medical records database.
